SEQUENCE LISTING (41 sequences)

SEQ ID NO: 1 - protein, 5 aa, Zea mays
YWLVK

SEQ ID NO: 2 - protein, 9 aa, Zea mays
SKGAVTPIK

SEQ ID NO: 3 - protein, 17 aa, Zea mays
PTTVMAWFILVPHCPEK

SEQ ID NO: 4 - protein, 7 aa, Oryza sativa
KGSTSVK

SEQ ID NO: 5 - protein, 9 aa, Oryza sativa
KQIMWSELS

SEQ ID NO: 6 - protein, 14 aa, Oryza sativa
AVWSALSTGEKQQR

SEQ ID NO: 7 - protein, 11 aa, Triticum aestivum
VVGGGGAVRGR

SEQ ID NO: 8 - protein, 5 aa, Zea mays
WFTSR

SEQ ID NO: 9 - protein, 7 aa, Zea mays
YLEQVLR

SEQ ID NO: 10 - protein, 10 aa, Zea mays
NLPVGDLVDK

SEQ ID NO: 11 - DNA, 19 bp, Artificial Sequence
  Description of Artificial Sequence Synthetic primer
cgactgcgacccctacgac

SEQ ID NO: 12 - DNA, 20 bp, Artificial Sequence
  Description of Artificial Sequence Synthetic primer
cgactgcgacccctacgacg

SEQ ID NO: 13 - DNA, 21 bp, Artificial Sequence
  Description of Artificial Sequence Synthetic primer
cgactgcgacccctacgacgg

SEQ ID NO: 14 - DNA, 22 bp, Artificial Sequence
  Description of Artificial Sequence Synthetic primer
cgactgcgacccctacgacggc

SEQ ID NO: 15 - DNA, 19 bp, Artificial Sequence
  Description of Artificial Sequence Synthetic primer
tctgcccccacgagttctt

SEQ ID NO: 16 - DNA, 20 bp, Artificial Sequence
  Description of Artificial Sequence Synthetic primer
tctgcccccacgagttcttg

SEQ ID NO: 17 - DNA, 21 bp, Artificial Sequence
  Description of Artificial Sequence Synthetic primer
tctgcccccacgagttcttga

SEQ ID NO: 18 - DNA, 22 bp, Artificial Sequence
  Description of Artificial Sequence Synthetic primer
tctgcccccacgagttcttgac

SEQ ID NO: 19 - DNA, 20 bp, Artificial Sequence
  Description of Artificial Sequence Synthetic primer
cggcatacgagaagtggttc

SEQ ID NO: 20 - DNA, 21 bp, Artificial Sequence
  Description of Artificial Sequence Synthetic primer
cggcatacgagaagtggttca

SEQ ID NO: 21 - DNA, 22 bp, Artificial Sequence
  Description of Artificial Sequence Synthetic primer
cggcatacgagaagtggttcac

SEQ ID NO: 22 - DNA, 23 bp, Artificial Sequence
  Description of Artificial Sequence Synthetic primer
cggcatacgagaagtggttcacg

SEQ ID NO: 23 - DNA, 20 bp, Artificial Sequence
  Description of Artificial Sequence Synthetic primer
agtggacccagaaccatgag

SEQ ID NO: 24 - DNA, 21 bp, Artificial Sequence
  Description of Artificial Sequence Synthetic primer
agtggacccagaaccatgagg

SEQ ID NO: 25 - DNA, 22 bp, Artificial Sequence
  Description of Artificial Sequence Synthetic primer
agtggacccagaaccatgagga

SEQ ID NO: 26 - DNA, 23 bp, Artificial Sequence
  Description of Artificial Sequence Synthetic primer
agtggacccagaaccatgaggac

SEQ ID NO: 27 - DNA, 20 bp, Artificial Sequence
  Description of Artificial Sequence Synthetic primer
ctgttgttcgtgaggcacat

SEQ ID NO: 28 - DNA, 21 bp, Artificial Sequence
  Description of Artificial Sequence Synthetic primer
ctgttgttcgtgaggcacatg

SEQ ID NO: 29 - DNA, 22 bp, Artificial Sequence
  Description of Artificial Sequence Synthetic primer
ctgttgttcgtgaggcacatga

SEQ ID NO: 30 - DNA, 23 bp, Artificial Sequence
  Description of Artificial Sequence Synthetic primer
ctgttgttcgtgaggcacatgac

SEQ ID NO: 31 - DNA, 20 bp, Artificial Sequence
  Description of Artificial Sequence Synthetic primer
tgaagctgttgttcgtgagg

SEQ ID NO: 32 - DNA, 21 bp, Artificial Sequence
  Description of Artificial Sequence Synthetic primer
tgaagctgttgttcgtgaggc

SEQ ID NO: 33 - DNA, 22 bp, Artificial Sequence
  Description of Artificial Sequence Synthetic primer
tgaagctgttgttcgtgaggca

SEQ ID NO: 34 - DNA, 23 bp, Artificial Sequence
  Description of Artificial Sequence Synthetic primer
tgaagctgttgttcgtgaggcac

SEQ ID NO: 35 - DNA, 1806 bp, Cynodon dactylon
  CDS (51)..(1091)
gatgcaaaagcagagaacatagtagaaacacctacgattacagttccattatggattccccccgcggagccacgtcggtgtttgctctaatcctgctctcttgcattctcatgttcgctgcctctccggtggcaacagggacgacggagggcgtggacgccgccggtgacaagcttatgatggaccggttcctccgctggcaggcagcacacaaccggtcctacccgagtgaggaggagaagcggcgtcggttcgaggtgtaccgccggaacgtggagtacatcgaggagaccaaccaggttggcaacctgacgtaccagctcggcgagaaccagttcacggacctgacgccggaggagttcctggacatgtacaccatgaaggggccagtgcatgacgagaaacccaccaacgtgtccttctctgagggcgtcgccgtggacgccccgaccagcgtggactggaggtcccaaggcgccgtgacgccgatcaagaaccaaggcccatcgtgctcaagctgctgggcttttgtgacgaccgcgacgatcgagagcctgagcaagatcagaacggggaggctggtctcgttatcggagcaggagctcatcgactgcgacccctacgacggcggctgcaacctgggctacttcgtgaacggttaccggtgggtcatcgagaacggcggcctcacgaccgacgccaactacccgtaccaggcgcgccgcaactactgcagccgttcccgggcgtccaactacgcctcccggatccgggactacgtgcaggtgccgtccggcgaggcggagctgcagcgtgcggtggtgcagcagcccgtggccgcggccgtcgagatgggcggcaacctgcagtactacagcggcggcgtcttctccggtcagtgcgggacgcggatgaaccacgccatcaccgtcgtcgggtacggcgccgacgcggccaccgggctcaagtactggatcgtcaagaattcgtgggggaccagctggggcgagcgcgggtacctgcggatgcgccgtgacgtcgggcgcgggggcatgtgcggcatcgcgctcgacctcgcgtacccgatcatgtaacaacgtgtcacgacacacgatgacgatgttactgtatctctaattaagctgccgcctctctaagcaccgccggctatgacgacgctgggagaaatgcgtgagcttggtatatggtttgtgtacttgtgtcctgcgtgcgttatgaaacaatctcttcgatcaaatatcatatagtatagtacgattaatgtgatatgtttctagctaaatgatgtttgttgtgtcttgtgttataaaacaatggccaatcaatacccaattagtacaattaaataaatgtaatatgtttctaaatgatgttcagtgataatcagcattaagctgtcacactaagtgcaagctatcgacaacaatgttcctttttaccttcaatgcaagaaattatacgattagtgaagaaaattcagtgacatacaacttttccatcgttaaacatgatgaaagcacataaaagtgtgttcgtatctaaatgaacgaaaccacagatatttatgacgaattctcatcataaaagtgtgaattcagtcccaccaatcgggaccatggtagtgaataatttatttcacttaatcatttctcacagggctagtcatagcttatagcttaagcattttcttatttactcaaattgatgactgcatagatatatctatgaacactcatttaatttactgtcatataatattagtggcatatttatcatgaaataggg

SEQ ID NO: 36 - protein, 347 aa, Cynodon dactylon
MDSPRGATSVFALILLSCILMFAASPVATGTTEGVDAAGDKLMMDRFLRWQAAHNRSYPSEEEKRRRFEVYRRNVEYIEETNQVGNLTYQLGENQFTDLTPEEFLDMYTMKGPVHDEKPTNVSFSEGVAVDAPTSVDWRSQGAVTPIKNQGPSCSSCWAFVTTATIESLSKIRTGRLVSLSEQELIDCDPYDGGCNLGYFVNGYRWVIENGGLTTDANYPYQARRNYCSRSRASNYASRIRDYVQVPSGEAELQRAVVQQPVAAAVEMGGNLQYYSGGVFSGQCGTRMNHAITVVGYGADAATGLKYWIVKNSWGTSWGERGYLRMRRDVGRGGMCGIALDLAYPIM

SEQ ID NO: 37 - protein, 161 aa, Sorghum halepense
  MOD_RES (53): Variable amino acid
DCDPYDGGCNLGYFVNGYRWVIENGGLTTEASYPYQARRNYCSRSKAAQHAAXISDYVQVXAGEGNLQQAVXQQPVAAAIEMXGSLQFYXGGVFSGQCGTRMNHAITVVGYGAEANTGLKYWIVKNSWGQSWGERGFLRIRRDATRSGLCGIALDLAYPVV

SEQ ID NO: 38 - protein, 19 aa, Sorghum halepense
WVIQNGGLTTEANYPYQAR

SEQ ID NO: 39 - protein, 24 aa, Sorghum halepense
QLHVVGDGGGVLGGPGAAARVPPR

SEQ ID NO: 40 - protein, 5 aa, Sorghum halepense
YWIVK

SEQ ID NO: 41 - protein, 161 aa, Sorghum halepense; identical to SEQ ID NO: 37
  MOD_RES (53): Variable amino acid

(19) United States
(12) Patent Application Publication
(10) Pub. No.: US 2009/0068233 A1
(43) Pub. Date: Mar. 12, 2009

(54) METHODS AND COMPOSITIONS FOR DIAGNOSIS AND IMMUNOTHERAPY OF POLLEN ALLERGY

(75) Inventors: Katinka A. Vigh, Cambridge (GB); Ravishankar Palanivelu, Tucson, AZ (US); Matthew A. Cummings, Chicago, IL (US); Jason Ward, Chicago, IL (US); Daphne Preuss, Chicago, IL (US)

Correspondence Address: BARNES & THORNBURG LLP, P.O. BOX 2786, CHICAGO, IL, 60690-2786, US

(73) Assignee: THE UNIVERSITY OF CHICAGO, Chicago, IL (US)

(21) Appl. No.: 12/281,166

(22) PCT Filed: Mar. 2, 2007
(86) PCT No.: PCT/US07/63188

§ 371 Date: Nov. 11, 2008

Related U.S. Application Data
(60) Provisional application No. 60/778,246, filed on Mar. 2, 2006.

(51) Int. Cl. A61K 39/00; C12N 9/50; C40B 50/14; C40B 30/04; A61P 37/00; C40B 30/00; C40B 40/10

(52) U.S. Cl. 424/275.1; 435/219; 506/30; 506/7; 506/18; 506/9

(57) ABSTRACT

A diagnostic pollen array includes allergens extracted from pollen coat material. Diagnostic pollen arrays are useful to diagnose allergy in individuals, identify novel allergens, identify genetic loci responsible for allergy in hosts, and to develop treatment plans for allergy.

### BACKGROUND

Current allergy diagnostics, such as skin prick (SPT) or radioallergosorbent (RAST) tests, typically rely on proteins soluble in aqueous buffers that are extracted from washed and lysed pollen grains. Similar extraction methods are often used to provide reagents for immunotherapy, a treatment for long-term allergy relief.

Knowledge of animal host genes that contribute to allergy susceptibility could facilitate the development of new, more effective allergy treatments for patients. An understanding of pollen components or pollen materials can provide important clues to their role in triggering allergy and asthma. The walls of pollen grains are composed of an unusual polymer coated with a lipid-rich layer that contains a small set of proteins. When this layer or surface contacts female flower cells, it rapidly diffuses; enzymes contained within this surface layer have the potential to alter pollen contents, as well as the composition of cell surfaces they contact. Pollen grains can absorb water from cells they contact and begin releasing intracellular enzymes that enable the extension of a pollen tube that carries sperm.

### SUMMARY

Pollen allergen arrays described herein accurately assess patients' sensitivity to a wide range of pollen allergens, including organic extracts from unwashed pollen. Non-pollen allergens, e.g., mite, dust, dander and the like can also be included in the diagnostic microarrays disclosed herein. Patient sensitivity to pollen allergen data, collected by screening individuals using a pollen array, define a specific and semi-quantitative pollen sensitization phenotype for identifying pollen susceptibility genes, either with linkage mapping or association studies.

One method for identifying genes that contribute to allergy is to correlate patient responses with genomic DNA markers. The pollen array described herein can be used to analyze sera from any group of patients, by measuring the presence of antibodies or any suitable immunological marker, to specific pollen components. When combined with genetic information, this phenotype data can be used to map or identify allergy susceptibility genes.

Compositions and methods relate extraction of pollen components and use of such extracted pollen components to diagnose allergen sensitization, to identify novel gene products in pollen, to identify allergy-specific genetic markers in hosts, and to develop allergy treatments. A collection of extracted pollen components including organic solvent extraction, for example, in an array format disclosed herein, diagnoses allergen sensitization in one or more individuals.

Pollen extracts routinely used for allergy diagnostics and therapy do not contain components extracted with organic solvents from unwashed pollen. Pollen arrays described herein include pollen components extracted from multiple allergenic species and are prepared by extraction of pollen components from unwashed pollens using an organic solvent. Pollen arrays described and disclosed herein include pollen allergens that are absent from commercially available traditional pollen extracts. Pollen arrays described and disclosed herein include pollen allergens from organic extracts and aqueous extracts of pollen components.

Pollen extracts disclosed herein are used for several purposes that include (i) providing a non-invasive diagnostic tool to measure a patient's sensitivity to pollen allergens; (ii) collecting patient response data that facilitate identification of genes that contribute to allergy; (iii) identifying and purifying novel allergens; and (iv) measuring patient IgE, IgA, and IgG antibodies.

The pollen material or component extraction and isolation methods described herein improve the accuracy of allergy diagnosis and the effectiveness of allergy therapy. The extraction and isolation methods disclosed herein maximize the capture of pollen allergens that otherwise would be lost in a traditional isolation and extraction procedure. For example, the capture of pollen surface allergens that include pollen coat proteins, lipids, or pollen wall material is enhanced. The extraction and isolation methods disclosed herein are designed to capture intracellular, extracellular, cell wall bound, and membrane bound pollen components from washed and unwashed pollens that may contribute to allergy.

A diagnostic pollen array is designed, for example, by applying or affixing pollen components to a solid support. A pollen array can include any form of support, such as for example, glass, plastic, polymer, epoxy-coated glass, nylon or nitrocellulose membrane or any similar support or substrate capable of bonding to allergens. An array as used in herein encompasses any ordered arrangement of pollen allergens or combinations of allergens. The ordered arrangement can include a low density array that may contain at least one pollen allergen, or a high density array that contains a plurality of pollen allergens. An array may also contain allergens in duplicates or multiple replicates, allergens that are derived from sources other than pollen, and may also contain allergenic and non-allergenic controls. Components may be spotted on a support or applied or affixed in any manner dependant on the type of support.

Allergens as used herein include proteins, peptides, lipids, carbohydrates, a combination thereof, or any biochemical factor capable of triggering a measurable allergy response. Allergens may also include both allergens purified to homogeneity, as well as allergens in crude extracts that contain mixtures of allergenic and non-allergenic components. These biochemical factors or allergens, or fragments thereof, can be intracellular, extracellular, membrane bound, or cell wall bound. Few pollen allergens have been directly tested for their ability to cause allergy and the possibility exists that they could merely cross react with commonly-produced IgEs or play secondary, but not causative roles. Molecules (peptides, proteins, lipids, carbohydrates or a combination or a fragment thereof) identified following the methods disclosed herein to cause allergenic responses are tested for pulmonary and cellular responses. A mouse model is a suitable system considered representative of human allergy. Promising molecules are tested in vivo by examining the ability of the allergens to provoke airway inflammation.

“Array or microarray” relates to molecules generally connected to a matrix or support (substrate) in a specific arrangement relative to each other.

“Component” as used herein means an element forming part of a whole and “material” as used herein means a constituent of an element of a substance.

A diagnostic pollen array described herein includes a pollen allergen selected from a group that includes pollen coat, cell wall-bound, membrane-bound, intracellular, and extracellular pollen material. “Pollen allergen” includes a biological component selected from peptides, proteins, lipids, carbohydrates, a fragment thereof, and a combination thereof. A pollen allergen in the array disclosed herein may also be present as a pollen extract. A pollen allergen is substantially purified. A pollen allergen may be a recombinant protein. A pollen allergen is present in an amount sufficient to detect an allergy response in a patient sample. The pollen allergen is selected from a washed pollen and/or an unwashed pollen.

The pollen allergen is obtained from an allergenic plant species. Preparations from several individual pollen species, spotted in either a high or low-density format, are included in the arrays. Sera from allergic or non-allergic patients are applied to the array, and the presence of antibodies is measured, providing an assessment of the patient's sensitivity to specific pollen species.

An allergenic plant species is selected from grasses, weeds, and trees. The allergenic plant species is selected from Wal (Walnut), Ber (Bermuda grass), Syc (Sycamore), Orc (Orchard grass), Pec (Pecan), Tim (Timothy grass), Olv (Olive), Mug (Mugwort), WOk (White Oak), Rag (Ragweed), Ald (Alder), Eld (Box Elder), Cot (Cottonwood), Mul (Mulberry), Jhn (Johnson grass), Elm, Ash, Ced (Cedar), Blu (Bluegrass), Bir (Birch), Rye, and ROk (Red Oak).

A pollen allergen is extracted using a solvent. The solvent is selected from a group of solvents that includes organic and inorganic solvents. The solvent is selected from a group that includes polar, non-polar, protic, and aprotic solvents. An organic solvent is selected from a group of solvents that includes for example, cyclohexane, hexane, diethylether, formamide, dimethylformamide, diemthyl sulfoxide, acetone, toluene, benzene, any suitable hydrocarbon solvents or halogenated solvents.

A pollen allergen may also be extracted using any suitable method that captures a pollen coat, cell wall-bound, membrane-bound, intracellular, and extracellular pollen material. Pollen extract materials can also be obtained or extracted using reagents from commercial suppliers, using their proprietary or FDA-approved methods so long as the reagents and the methods enable isolation of all the pollen components and fractions disclosed herein.

A method of diagnosing allergy in a sample from an individual, includes the steps of:

(a) providing a diagnostic pollen array;

(b) applying the sample from the individual to the pollen array;

(c) measuring antibody reactivity in the sample compared to a control; and

(d) diagnosing allergy in the individual.

The sample includes blood and serum and the antibody reactivity may be measured using a detectable label.

A method of identifying a genetic locus that contributes to allergy, includes the steps of:
(a) providing a diagnostic pollen array;
(b) diagnosing allergen sensitization in a group of individuals;
(c) correlating the allergy response in the group of individuals to at least one genetic marker; and
(d) identifying the genetic locus that contributes to allergy.

The genetic marker includes a DNA marker. The group of individuals comprises a subpopulation that is genetically related. The correlation of the allergy response is through segregation analysis and linkage mapping of a group of individuals whose pedigrees are known. For association mapping the individuals do not have to be related.

A method to measure antibody levels to pollen allergens in an individual, includes the steps of:
(a) providing a diagnostic pollen array;
(b) providing a serum sample from the individual; and
(c) applying the serum sample from the individual to the diagnostic array; and
(d) measuring antibody levels, where the antibody levels include at least one of IgE, IgA, and IgG antibodies.

A method to develop a pollen-specific allergy treatment in an individual, includes the steps of:
(a) providing a diagnostic pollen array;
(b) identifying a pollen allergen that contributes to allergy in the individual; and
(c) developing a therapy to treat allergy to the pollen allergen in the individual, where the therapy includes identifying and administering a pharmaceutical composition.

A method to identify novel pollen allergens, includes the steps of:
(a) providing a diagnostic pollen array;
(b) identifying a pollen allergen that reacts with a serum sample obtained from at least one allergic individuals;
(c) obtaining sequence information of the pollen allergen, where the sequence information may be obtained through, for example a mass-spectroscopic analysis.

Using the methods described for the identification of novel allergens provides new and potentially more effective immunotherapy treatments, for example, the use of a cysteine protease to treat allergy by desensitizing allergic patients to purified, recombinant cysteine protease. Additionally the use of an inhibitor of a cysteine protease to prevent the symptoms of allergy or asthma is a treatment option is contemplated.

For treatments of allergy such as immunotherapy, where individuals are desensitized with the actual allergens to which they are reactive, all allergen components which contribute to the individual's allergy must be correctly identified. Thus, the allergen array facilitates more effective treatments by more accurately and comprehensively diagnosing allergy. Additionally, identification of allergy susceptibility genes is facilitated by the use of the allergen array as a method for phenotyping a population. Identification of such genes can directly and indirectly identify potential drug targets. These drug targets are then guides in the development of appropriate treatments for allergy.

### BRIEF DESCRIPTION OF THE DRAWINGS

The drawings are provided to illustrate some of the embodiments of the disclosure. It is envisioned that alternate configurations of the embodiments of the present disclosure are within the scope of the disclosure.

FIG. 1 represents a low-density pollen array. Components extracted from washed pollen were blotted in duplicate spots onto nitrocellulose and probed with antisera from non-allergic (1) or allergic (2, 3) individuals (Left). Sensitivity to four pollen species was tested: Tim (Timothy grass), Rag (Ragweed), WOk (White Oak) and Olv (Olive). Dark spots indicate the individual sera contain IgE antibodies that react with the pollen material. Components extracted from washed (W) or unwashed (UW) pollen were blotted in duplicate spots onto nitrocellulose and probed with antisera pooled from 500 individuals (Catalog #HMSRM, Bioreclamation Inc., Hicksville, N.Y.). (Right) 22 pollen species were tested, including Wal (Walnut), Ber (Bermuda grass), Syc (Sycamore), Orc (Orchard grass), Pec (Pecan), Tim (Timothy grass), Olv (Olive), Mug (Mugwort), WOk (White Oak), Rag (Ragweed), Ald (Alder), Eld (Box Elder), Cot (Cottonwood), Mul (Mulberry), Jhn (Johnson grass), Elm, Ash, Ced (Cedar), Blu (Bluegrass), Bir (Birch), Rye, and ROk (Red Oak). Controls in which the human sera or the secondary anti-IgE antibody were spotted directly onto the nitrocellulose were also included.

FIG. 2 shows a layout of a diagnostic pollen array containing commercially available pollen extracts, cytoplasmic and surface pollen extracts prepared according to the methods disclosed herein, commercially available non-pollen extracts, negative controls and standard IgA, IgE, and IgG antibodies.

FIG. 3 shows images of scanned allergen arrays that are incubated with sera from individual 1 (A) or individual 2 (B) and antibody binding is detected with fluorescently labeled anti-IgE.

FIG. 4 shows comparison of IgE standard curves that demonstrate reliability and accuracy of data analysis on diagnostic pollen arrays.

FIG. 5 is a graph showing pollen reactivity profiles for 3 individuals. Reactivity to cytoplasmic and surface components from 5 pollen is shown as different hatchings and shadings. Bound IgE is extrapolated from the median fluorescent intensity of the IgE standard curves.

FIG. 6 is a photographic reproduction showing that treatment of Bermuda grass pollen with organic solvents alters the surface layers. Exine channels, indicated by arrows, appear to collapse after organic solvent washes. The close proximity of the exines' dark staining inner channel surfaces makes the collapsed channels of the washed pollen appear darker than those of the untreated pollen. E, exine; I, intine; C, cytoplasm.

FIG. 7 shows gel images of allergens isolated from the surface and cytoplasmic fractions of Bermuda grass pollen proteins. Stained image (A) and western blot (B) show distinct bands.

FIG. 8 shows the identity of some of the allergens from the Bermuda grass pollen extract. The IgE binding proteins were identified via peptide fingerprinting on a matrix assisted laser desorption/ionization-time of flight (MALDI-TOF) instrument.

FIG. 9 shows identities of IgE-binding proteins from the cyclohexane-washed fractions are confirmed by 2D western blot analysis. (A) Bermuda grass cyclohexane-washed fraction was separated by 2D PAGE and stained with Sypro Ruby (Bio-Rad). (B) A Western blot analysis of the 2D PAGE gel in (A) was performed by probing with sera pooled from 500 individuals and an IgE-specific secondary antibody. The identities of spots E1, E2, and C1 as two endoxylanases and a cysteine protease were verified by mass-spectrophotometry peptide ID. The position of molecular weight markers is shown at the left. IgE in the pooled sera also bound the 14.4 kDa mass marker, lysozyme from chicken egg white.

FIG. 10 Cloning of the IgE-reactive cysteine protease from Bermuda grass. The full-length cDNA sequence was cloned from Bermuda grass floral RNA. The protein sequence appears below the mRNA open reading frame. The numbers on the left indicate the nucleotide position at the beginning of each line. The numbers on the right indicate the amino acid position at the end of each line. The arrows indicate the putative cleavage site for the signal peptide (top arrow) and the cleavage site of the pro-domain (bottom arrow). Sequences that match peptide-sequencing results of the IgE-reactive band are shaded in gray. Sequences that do not match peptide-sequencing results are shaded in black.

FIG. 11 Individual sera show significant IgE reactivity to Bermuda grass pollen cyclohexane-extracted proteins. Cyclohexane-extracted fractions were separated by SDS-PAGE and Western blot was performed with sera either pooled from 500 random individuals (P) or serum from individuals with high titers of IgE (1-7), and a secondary antibody to IgE. The position of molecular weight markers is shown at the left.

FIG. 12 Cyclohexane-extracted pollen fractions from other grass species contain IgE-reactive proteins. The left panel for each grass is a SDS/PAGE of proteins stained with Coomassie G250. Asterisks denote IgE binding proteins. The right panel for each grass is a western blot probed with sera pooled from 500 individuals. IgE binding proteins were identified via mass-spec peptide identification. The position of molecular weight markers is shown at the left.

FIG. 13 Cloning of the Johnson grass surface cysteine protease. The amino acid sequence translated from a partial cDNA sequence cloned from Johnson grass floral RNA. Sequences that match peptide-sequencing results of the IgE-reactive band are shaded in gray. Sequences that do not match peptide-sequencing results are shaded in darker gray.

FIG. 14 Amino acid alignment of Bermuda grass and partial Johnson grass cysteine protease sequences identified from pollen surface fractions. The alignment was generated using ClustalW. *=identical amino acids, :=strongly similar amino acids, and .=weakly similar amino acids.

FIG. 15 shows comparison of pollen cytoplasm extracts isolated by the methods disclosed herein with a commercially available pollen extract (Greer's) using polyacrylamide gel electrophoresis for birch and red oak.

FIG. 16 shows IgE binding protein band from a pollen extract of Pecan.

FIG. 17 A. Images from scanned arrays showing reactivity of serum from individual 85806 (right panel) as compared to a blank array (left panel) to commercial extracts (e), cytoplasmic proteins (c), and surface proteins (s) from 10 pollen species. Note: Signals on blank array due to autofluorescence of proteins. B. Quantification of reactivity to pollens described in A. Bars represent the mean (n=18 arrays) with standard deviation error bars.

### DETAILED DESCRIPTION

Methods and compositions relating to diagnostic pollen arrays involve developing arrays that include allergens from unwashed pollen, the pollen surface or pollen coat. The pollen arrays disclosed herein that contain both intracellular, extracellular and pollen coat-specific allergens, are useful to diagnose allergen sensitization with an increased level of accuracy and specificity, identify associated or linked host genetic markers and develop treatment plans that are targeted towards particular allergy responses.

Diagnostic pollen arrays described herein are constructed by selecting allergenic pollen species, collecting and extracting pollen components or pollen extracts from the pollen and constructing variable density pollen arrays.

Allergenic pollen species are selected through available knowledge in the literature. For example, a wide range of allergenic pollen species are known, including grasses, weeds, and trees. The OPAL™ database (Ogren, 2000) indicates the extent of pollen allergenicity from a wide range of species, with a score of 10 being the most allergenic. Components from pollen described in this database, as well as from other pollen species, are extracted as disclosed herein.

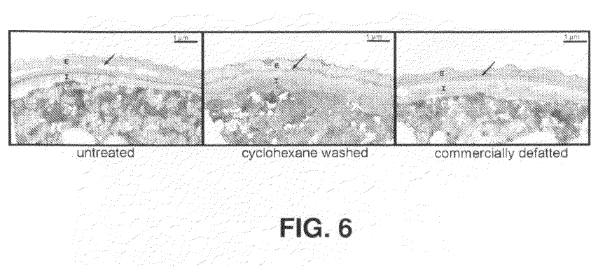

Pollen surface proteins from Arabidopsis thaliana can be removed without causing the pollen grain to hydrate or release its cytoplasmic contents by washing the pollen with the organic solvent cyclohexane. Since commercial pollen extracts are routinely washed with an organic solvent, the surface layers of Bermuda grass pollen may be altered by this treatment. Transmission electron micrographs (TEMs) confirm that washing pollen with cyclohexane or acetone (commercially defatted) does not rupture the grain or disrupt the cytoplasm (FIG. 6). However, these TEMs show the channels within the exine have collapsed after organic solvent washes. The close proximity of the dark staining inner channel surfaces of the collapsed exine make these channels appear darker than those of the untreated pollen. The exine channel collapse is likely induced by the removal of the pollen coat or pollenkit, a lipid and protein rich matrix.

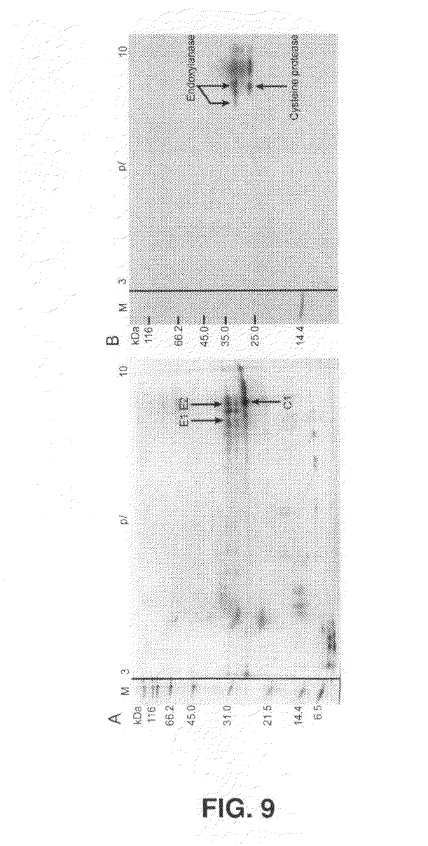

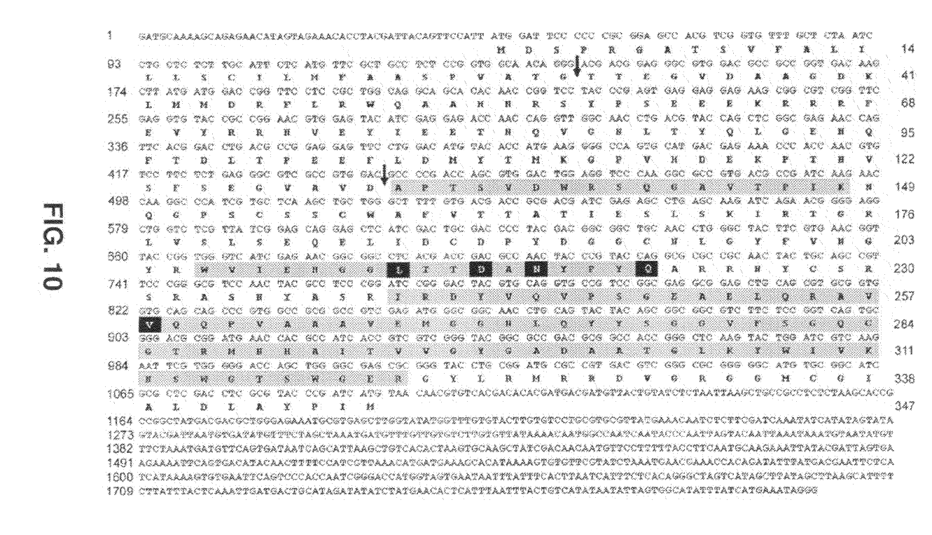

To verify the identity of the IgE-binding proteins, a 2D gel and corresponding western blot of Bermuda grass cyclohexane extracted proteins was performed (FIG. 9). The identities of two protein spots thought to be isoforms of endoxylanse (FIG. 9) and one spot believed to be a cysteine protease were verified by mass-spec peptide identification. These three proteins spots were shown to bind human IgE via western blot (FIG. 9).

Since the putative cysteine protease had the most reactivity with IgE from pooled sera, the cDNA was cloned. The cysteine protease was cloned from cDNA made from Bermuda grass floral RNA. Floral RNA was used since surface proteins are synthesized and assembled onto the pollen by the tapetal cells of the flower. To clone the cDNA, degenerate primers were designed using the peptide sequences generated by sequencing the IgE-reactive band and from conserved sequences of similar cysteine proteases from Zea mays and Oryza sativa. These primers produced a PCR product of approximately 300 bp. This PCR product was sequenced and used to design primers for 5′ and 3′RACE, which was used to clone the full-length cDNA. The full-length cDNA is 1806 bp, and the largest open reading frame is 1044 bp, which encodes a 347 amino acid protein (FIG. 10).

The first 30 amino acids of the protein are predicted by SignalP3.0 to be signal peptide. Additionally, N-terminal sequencing showed that the protein contains a pro-domain of 101 amino acids. Once the pro-domain is cleaved (after amino acid 131), the mature protein is 216 amino acids with a calculated molecular weight of 23.6 kDa (FIG. 10). This is the approximate size of the native protein when separated by SDS-PAGE. The cloned cDNA encodes a protein that significantly matches peptide-sequencing results of the IgE-reactive cysteine protease (FIG. 9), and show significant homology to known cysteine proteases. Although peptide-sequencing results significantly match the cloned protein sequence, there are discrepancies between the two sequences (FIG. 10). In some cases these discrepancies are the result of poor sequencing results or may indicate the presence of multiple isoforms of this cysteine protease. The 2D gel results suggest that multiple isoforms of this cysteine protease exist (FIG. 9).

The cDNA encoding the 347 amino acid protein was cloned into an E. coli expression vector and recombinant protein has been purified. This recombinant protein was not IgE-reactive, suggesting proper folding or a post-translational modification is required for IgE recognition of this protein. This protein may be purified using a Baculovirus expression system by the methods known to those of skill in the art.

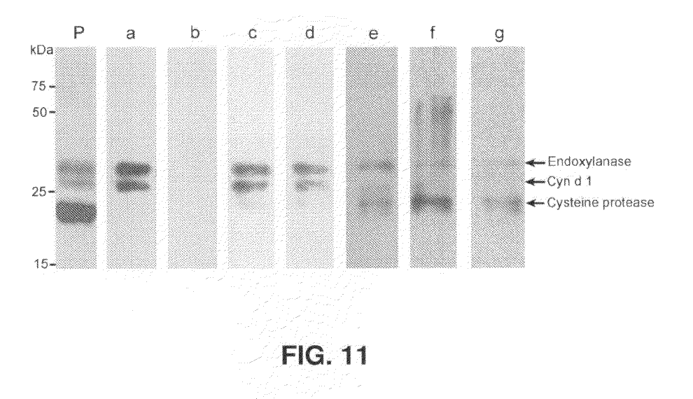

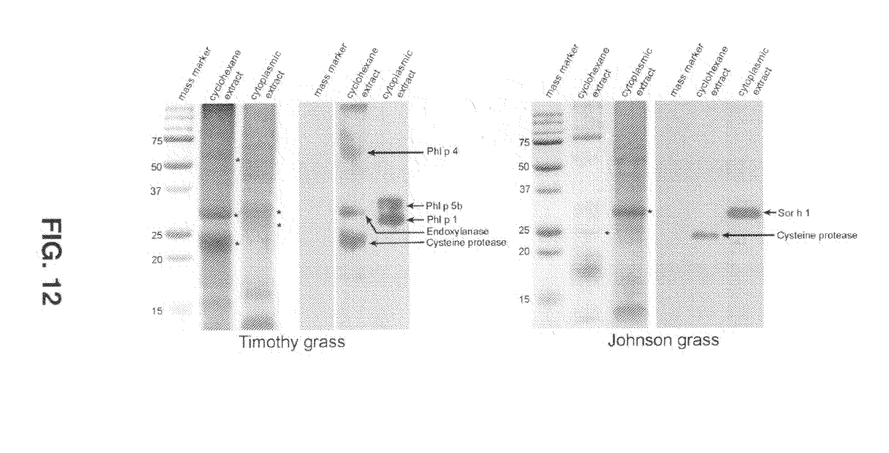

When sera pooled from multiple random individuals were used, the cysteine protease identified above had significant reactivity with IgE. To determine the extent of IgE binding in individuals, cyclohexane-extracted proteins were separated by SDS-PAGE and immunoblots for each of seven individual sera was performed (FIG. 11). These individuals were known to have high titers of IgE and showed IgE reactivity to Bermuda grass cytoplasmic proteins. Individual sera displayed differential binding to the IgE-reactive proteins identified by the pooled sera. Three individuals showed significant IgE-reactivity to the cysteine protease and little or no IgE-reactivity to Cyn d 1 (FIG. 11). Interestingly, six individuals had some IgE-reactivity to the endoxylanase, suggesting that Bermuda grass pollen proteins extracted by organic solvents show significant IgE-reactivity with allergy patient sera.

To determine if IgE-reactive cysteine proteases are present in plant pollens other than Bermuda grass, the immunoblots were repeated with cyclohexane-washed and cytoplasmic protein extracts from Timothy and Johnson grasses (FIG. 12). IgE-binding proteins were identified. IgE-reactive cysteine proteases were identified in both Timothy and Johnson grasses. These proteins were approximately the same size as the Bermuda grass cysteine protease. Peptide sequencing results show significant homology to the Bermuda grass protein, suggesting this family of cysteine protease may be cross reactive. An IgE-binding endoxylanase was also identified from the Timothy grass cyclohexane extracted fraction. In addition to these novel IgE-binding proteins a number of known allergens were identified including Sor1 form Timothy grass and Phl p 5b and Phl p 1 from Johnson grass.

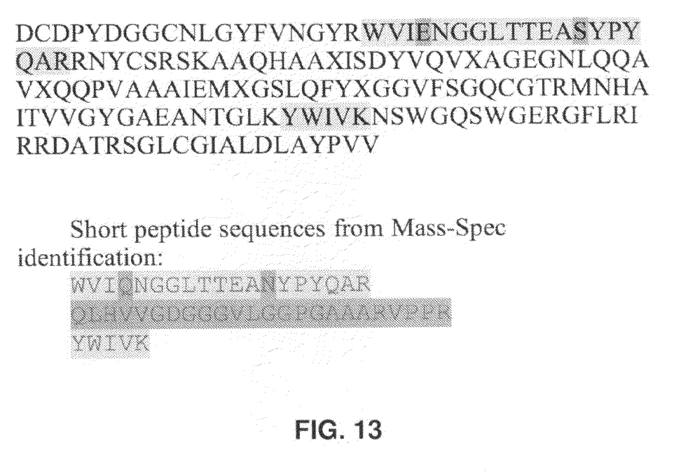

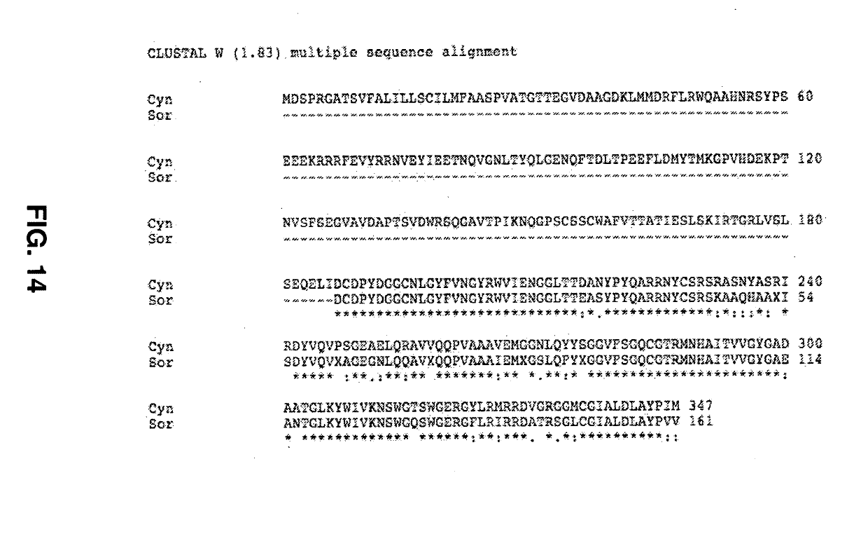

Johnson grass cysteine protease is cloned from a partial sequence from cDNA made from Johnson grass floral total RNA. The cloning protocol is identical to the one used for the Bermuda grass cysteine protease (FIG. 10). The partial cDNA sequence when translated significantly matches both the peptide sequence results generated from sequencing the IgE-reactive band (FIGS. 12 and 13) and also significantly matches the cloned Bermuda grass cysteine protease cloned (FIGS. 10 and 14).

### EXAMPLES

The following examples are illustrative and do not limit the scope of the various methods and compositions disclosed herein.

### Example 1

### Extraction of Pollen Coat Material

Extraction of pollen coat components is performed following the methods disclosed herein. To remove hydrophobic components, including those on the pollen surface, unwashed pollen was extracted with cyclohexane or any suitable organic solvent that is capable of extracting pollen coat components. Insoluble solid material is removed by centrifugation, and components dissolved into the organic phase are collected and concentrated by evaporation of the organic solvent under air or nitrogen. Residual components are precipitated with trichloroacetic acid (TCA), ammonium sulfate, acetone or other suitable reagents. To obtain components from the pollen cytoplasm, pollen washed with cyclohexane or other organic solvents such as dimethylformamide, carbon tetrachloride, or a combination thereof, is lysed in an aqueous buffer, using a mortar and pestle. Solid (insoluble) material is removed by centrifugation, and the soluble components are used directly, or are precipitated with trichloroacetic acid (TCA), ammonium sulfate, isopropanol or other suitable precipitating reagents. Precipitated components are dissolved in solutions containing potassium or sodium salts, buffered at a pH between 6 and 8, and containing about 5% or less of a non-ionic or an ionic detergent.

### Example 2

### Pollen Fractionation

Each pollen sample may initially be divided into three fractions: (a) extracellular components that are washed from the pollen grain with organic solvents as in Example 1 and as described in the Materials and Methods section; (b) components from lysed pollen grains that dissolve into aqueous buffers; and (c) insoluble components, including the pollen wall. For the extracellular fraction, proteins are separated from lipids based on their differential solubility in isopropanol and chloroform, the lipids are retained for GC/MS analysis. Intracellular fractions are used directly or are extracted with trichloroacetic acid, and/or polyvinylpolypyrrolidone and 2-mercaptoethanol and washed with cold acetone.

### Example 3

### Construction of Pollen Arrays

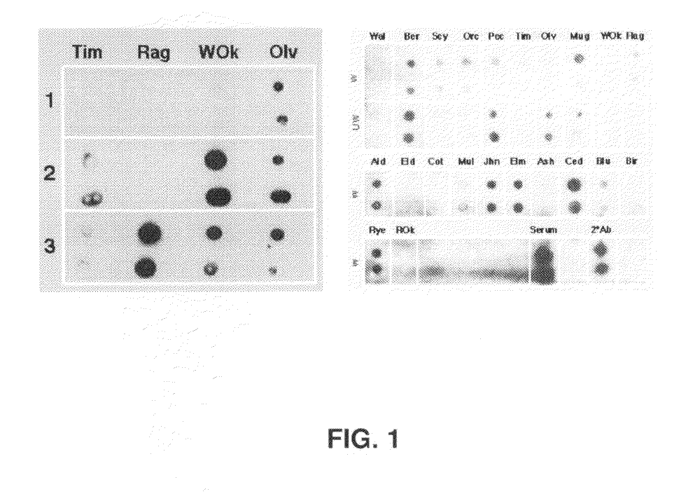

For a low-density pollen array (see Materials and Methods), approximately 1-10 μl of suspended pollen components, extracted as described in Example 1 or Example 2, are spotted onto nitrocellulose or any suitable membrane to form a low-density pollen array. Membranes can be blocked with proteins such as bovine serum albumin or non-fat milk and incubated with individual or pooled human sera following standard procedures. Bound antibodies are detected with labeled anti-human secondary antibodies, such as anti-IgE, anti-IgG, or anti-IgA. In the example illustrated in FIG. 1, bound human antibodies were detected with a horseradish perodixase (HRP) conjugated anti-human IgE secondary antibody and visualized with a luminescent substrate. The secondary antibody can be labeled with any detectable label. This experiment demonstrated distinct differences between patients in their IgE reactivity to pollen components, and sera pooled from 500 individuals demonstrated evidence of stronger sensitivity to components extracted from unwashed pollen, as compared to extracts from washed pollen.

For a high-density array (see Materials and Methods), pollen components can also be arranged on a support generally referred to as a microarray. Pollen components isolated from a variety of plant species can be arranged in duplicates or triplicates or in any other suitable format in a support. Generally, the support may be solid and includes glass, plastic, or any other suitable material. Spotting or affixing extracted pollen components can be performed using techniques known to a skilled artisan.

### Example 4

### High Density Allergen Arrays

A high-throughput high density protein microarray based assay to quantitatively measure allergen sensitization was developed. Pollen surface and cytoplasmic materials isolated from 22 highly allergenic plant species have been spotted in triplicates onto glass slides at a concentration of 0.3-1.0 μg/μL (ArrayIt™, Sunnydale, Calif.). Depending on the sensitivity of the solvents, labeling agents, signal scanner, signal analysis, and noise reduction, the concentration of the pollen materials or other allergen or non-allergen spotted on the array can be lower or higher than the ranges disclosed herein. For example, the concentration of pollen allergens can further range from about 10 nanograms to about 100 nanograms/μl or from about 1.0 μg-10.0 μg/μl. Commercially available extracts of the same pollens as well as 9 non-pollen and 5 recombinant allergens have also been included in the high density array. Unique reactivity in both the fractions prepared in the inventors' labs and the commercially available extracts has been observed. In addition to the allergens, 3 immunoglobulin standards were also spotted allowing for standardization of reactivity across different arrays. These are purified IgE, IgG, or IgA proteins—the primary antibody that the secondary antibody binds to. Quality and reproducibility of the allergen arrays disclosed herein are comparable or better than previous methods using recombinant proteins.

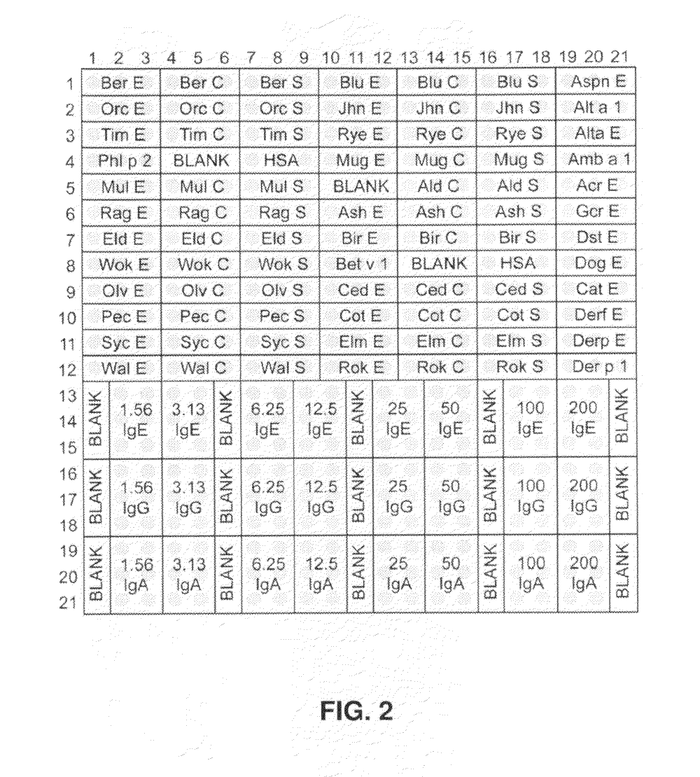

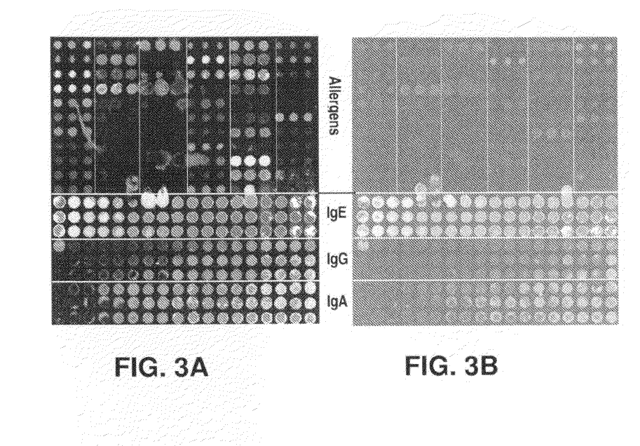

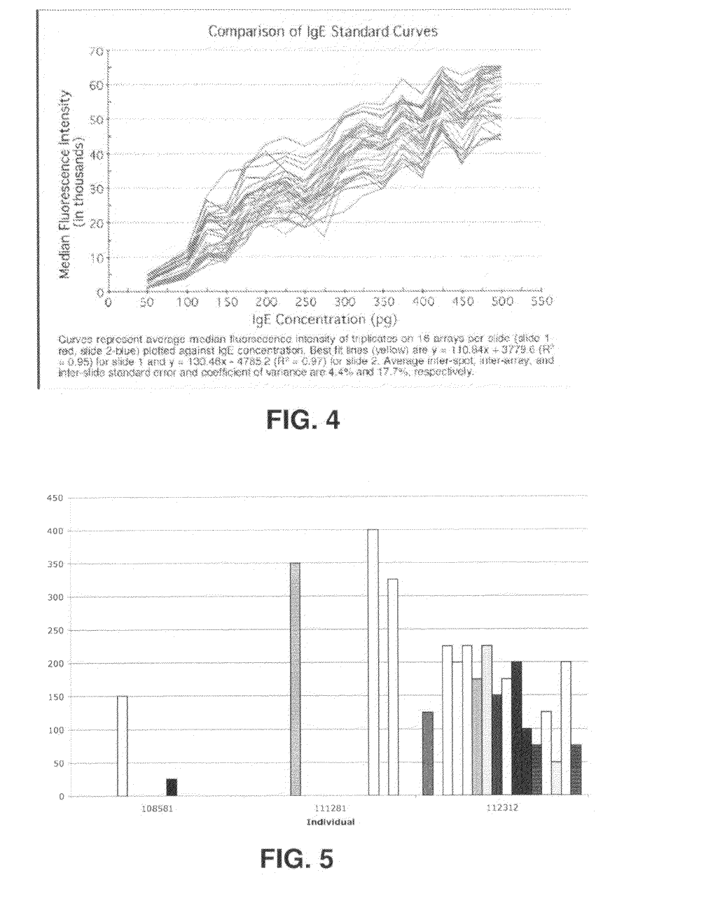

In the illustrated example on FIG. 3, a sample size of about 20 μl of human sera is sufficient to allow for the survey of reactivity to a wide range of pollen species, including all pollen components, and several non-pollen allergens. 80 different allergens can be tested at once. Allergen sensitization is detected with fluorescently labeled secondary antibody (anti human IgE, IgG1, IgG2, and IgA). Screening individual sera revealed distinct allergen sensitization profiles between individuals (FIG. 3), and commercial sera pooled from 500 people showed cases of stronger sensitization to surface as compared to cytoplasmic materials. FIG. 2 shows one embodiment of a layout of a diagnostic pollen array containing commercially available pollen extracts, cytoplasmic and surface pollen extracts prepared according to the methods disclosed herein, commercially available non-pollen extracts, negative controls and standard IgA, IgE, and IgG antibodies. The amount of the spotted standard antibodies is in picograms. FIG. 4 shows comparison of IgE standard curves that demonstrate reliability and accuracy of data analysis on diagnostic pollen arrays. Quality and reproducibility of the diagnostic pollen allergen array is comparable or better than previous methods using recombinant allergens. FIG. 5 shows pollen reactivity profiles of three individuals to various pollen species (both cytoplasmic and surface fractions). The sera from these individuals were screened against the diagnostic pollen array disclosed herein. The individuals show specific and distinct response to pollen extracts from various pollen species.

The allergen arrays disclosed herein are an effective way of assessing the allergen sensitization phenotype of individuals and aid in mapping allergy susceptibility genes. Screening a highly inbred religious isolate called the Hutterites aids in identifying the genetic links to allergy. Comparable numbers of an urban, more ethnically diverse population, such as Chicagoans, can also be screened using the allergen arrays disclosed herein. Using these quantitative and multidimensional allergen sensitization phenotype phenotype-genotype correlations to identify genes that predispose people to allergy are performed.

### Example 5

### Patient Sensitization to Pollen Allergens

When diagnosing patient sensitization to pollen allergens, arrays with extracted pollen material from multiple plant species disclosed herein offer advantages. For example, when patient responses to a large numbers of allergens are examined using a skin prick test, there is often significant skin irritation and discomfort. In contrast, only a few microliters of patient sera are required to probe a high-density array. Thus, specific and accurate allergic response to multiple pollen allergens can be obtained simultaneously using less sample volume and reducing discomfort to patients.

In an example illustrated in FIG. 2, pollen components extracted from washed and unwashed pollen were dissolved in a buffer containing salts and detergent and spotted in triplicate onto SuperEpoxy™ slides by a commercial arraying company (ArrayIt, Inc., Sunnyvale, Calif.). Any solid support capable of affixing proteins, tissues, cells, and lysates is suitable for the construction of the arrays disclosed herein. Three different immunoglobulin standards (IgE, IgG and IgA) were also spotted, as well as human serum albumin, and known recombinant allergens as controls. Following hybridization with human sera, bound antibodies were detected with fluorescently labeled anti-human IgE, IgG, or IgA. The quantity of bound antibody was measured by detecting fluorescence with a high-density detector.

### Example 6

### Mapping Pollen Allergy Genes

Diagnostic pollen arrays disclosed herein are used to assess the allergen sensitivity of individual patients. Individuals are categorized according to their pollen sensitization phenotypes including (1) reactivity to any pollen species, (2) reactivity to pollens from the same types of plants, such as grasses (including monocots) or dicots, and (3) reactivity to specific pollen species. Preferably, the entire study population may have more than 100 individuals, even more than 800 individuals, or more than 4000 individuals. As the size of the population increases, the ability to find statistically significant correlations between a particular genetic variant and susceptibility to pollen sensitization also increases.

A sample of DNA of sufficient quantity to perform the desired analysis can be acquired from each individual by any method known in the art. A suitable source and quantity of DNA for this purpose is 10-30 ml blood, since enough DNA can be extracted from leukocytes from such a sample to provide a sufficient quantity of DNA to perform many repetitions of any analysis contemplated herein.

A list of genetic variants is created that are used to map the pollen allergy phenotypes described above to specific locations in the genome. This list may come from a database of known genetic variants (e.g. dbSNP), may be generated through de novo polymorphism discovery in the study population, or may consist of some combination of the two.

After the list of genetic variants, or “markers”, is established, the variants can be analyzed for their correlation with pollen allergen sensitivity, or diseases related to pollen allergen sensitivity. DNA samples from individuals in the population are analyzed to determine which variants occur in each individual. This analysis can be performed using any method known in the art, including direct sequencing, RFLP methods, allele-specific PCR or SNP genotyping. The resulting catalog of patient genotypes are then correlated to pollen allergy phenotypes using statistical analyses such as linkage mapping or association mapping. In both methods, there is an implicit assumption that the phenotype of interest is either caused by genetic variation or that genetic variations affect the probability that an individual will manifest the phenotype.

Linkage mapping is well known to the skill in the arts and uses related group of individuals. In the simplest form of linkage analysis, two-generation families are collected which contain individuals with the phenotype of interest. At each genetic marker, the inheritance pattern of alleles through the family is compared to the inheritance pattern of the trait. The similarity of the two segregation patterns is quantified by the log odds of linkage or “LOD” score. The statistical properties of the LOD score are well-characterized and rules for declaring significance are well-known to those skilled in the art.

Association studies are conducted with families or with unrelated cases and controls. In brief, a statistical test is used on the distribution of genotypes among patients with a given pollen allergy phenotype (cases), relative to patients without that phenotype (controls), but matched for other variables, such as age, gender, and ethnicity. Where possible, multiple regression analysis can be used to determine interactions among any of the genetic variants. Isolated populations present a suitable case for both linkage and association studies due to the limited allele diversity and pedigree availability.

### Example 7

### Identification and Isolation of Novel Allergens

Diagnostic pollen arrays disclosed herein are used to identify and purify specific pollen allergens from mixtures extracted from unwashed or washed pollen. After individual or pooled sera are identified that react with pollen material, that pollen material is further fractionated. Various fractionation procedures are employed, including column chromatography, high pressure liquid chromatography, or electrophoresis. These fractions are spotted into a low or high-density array, and this new array is probed again with sera. Spots that react with the sera are further fractionated to homogeneity, and their components identified through protein sequencing, mass spectrometry, gas chromatography/mass spectrometry, NMR (nuclear magnetic resonance), or other analytical techniques well known to those of skill in the art.

Novel allergens detected in the pollen coat fractions are subjected to a proteomic analysis to identify peptide sequence and used to clone their respective genes. Sufficient material from the pollen coat of the allergenic species is isolated for detection of the relevant proteins on an acrylamide gel stained with Coomassie or other suitable reagents. Western blotting with patient sera is used to confirm the identity of the relevant band. Excision of the band, followed by digestion with specific proteases yields peptides that are sequenced using techniques known to those of skill in the art. After obtaining peptide sequences, GenBank and other relevant databases are searched to identify candidate genes or ESTs. Genes corresponding to completely novel peptides are cloned from cDNA libraries prepared from anthers at a series of developmental stages. Degenerate PCR primers that correspond to the sequenced peptides are used to amplify the pollen coat genes from these cDNAs. Subsequently, 5′ and 3′ RACE experiments are used to characterize the full-length message. A cDNA expression library from anthers at various developmental stages is also constructed to aid in the identification of novel pollen allergens that are reactive to patient sera.

Non-proteinaceous allergens including lipids are identified. Lipids from the pollen coating may serve as irritants that exacerbate the immune response. Lipids extracted from commercially prepared birch and timothy grass pollen induce polymorphonuclear granulocyte migration. The broad role of pollen lipids, particularly those from the pollen coating, has not been generally tested. Lipophilic molecules derived from plants cause contact inflammation and allergy; such molecules include urushiol, a phenolic lipid from poison ivy; and falcarinol, a 17-carbon alkene from English ivy.

Insoluble pollen material is also a source of important allergens. Much of this material is likely to be derived from the sporopollinen that comprises the exine wall, a polymer layer that may contain lipids, carotenoids, and phenolics. Exine walls contain species-specific adhesives. Insoluble material that reacts with sera from several patients is subjected to further analysis to identify the corresponding allergens as disclosed herein. For example, immunoelectron microscopy of pollen sections can ensure that the IgE signal is due to reactivity with exine, rather than from binding to cytoplasmic protein aggregates. Then, the ability of patient sera to cause purified wall fragments to aggregate confirms the presence of allergenic epitopes. Treatment of the exine fragments with different chemical regimes until their ability to bind IgE is destroyed indicates the chemical nature of the epitope. Solubilization of the exine, followed by NMR or mass spectroscopy may be required to identify the allergen. To facilitate this purification and identification process, a closely related ‘control’ pollen that is not recognized by the patient sera may be utilized.

### Example 8

### Treatment of Allergy Including Asthma

Diagnostic pollen arrays disclosed herein are useful to identify novel pollen allergens. Pollen is one of the most common triggers of asthma, along with dust mites, mold, and pets. Understanding pollen allergy remains an important health care problem, and understanding its causes represents a promising avenue for the prevention and treatment of asthma.

Most treatments for allergy and asthma require long-term, and often, daily medication to reduce histamine levels, mitigate inflammation and dilate bronchial passages. An improved understanding of the interactions between pollen grains and the cells that line pulmonary epithelia could lead to important advances. For example, the complex mixture of allergens on the pollen surface may directly signal immune cells, triggering previously by uncharacterized responses. New therapeutics could ultimately target such responses. In addition, the lipases that coat the pollen surface could alter the composition of mucosal layers themselves, thereby creating signals that stimulate allergenic response.

Immunotherapy remains a promising alternative in the treatment of pollen allergy. By focusing on compounds that reside on the pollen surface, the compositions and methods disclosed herein can lead to novel drug targets as well as to desensitization immunotherapy to novel groups of pollen allergens. Surveying IgE from the sera of several families, novel correlations that explain the inheritance of a predisposition towards allergy and asthma may be obtained. Immunotherapy with recombinant protein allergens, or with purified epitopes from the pollen wall and coating, may yield treatments with an improved efficacy that are significantly improved relative to the whole-cell pollen extracts in present use.

### Example 9

### Identification of Novel Pollen Allergens from Bermuda Grass Pollen Through Allergen Isolation, IgE Antibody Screening, and Sequencing

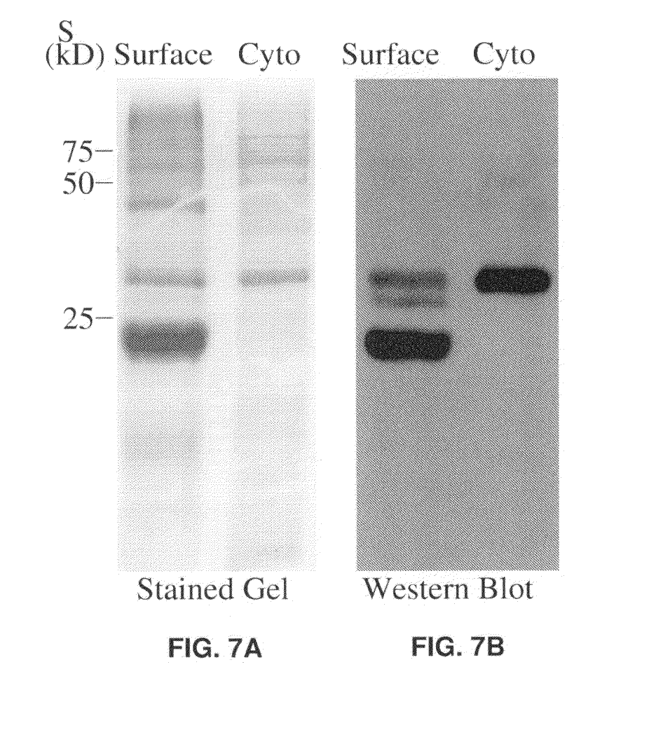

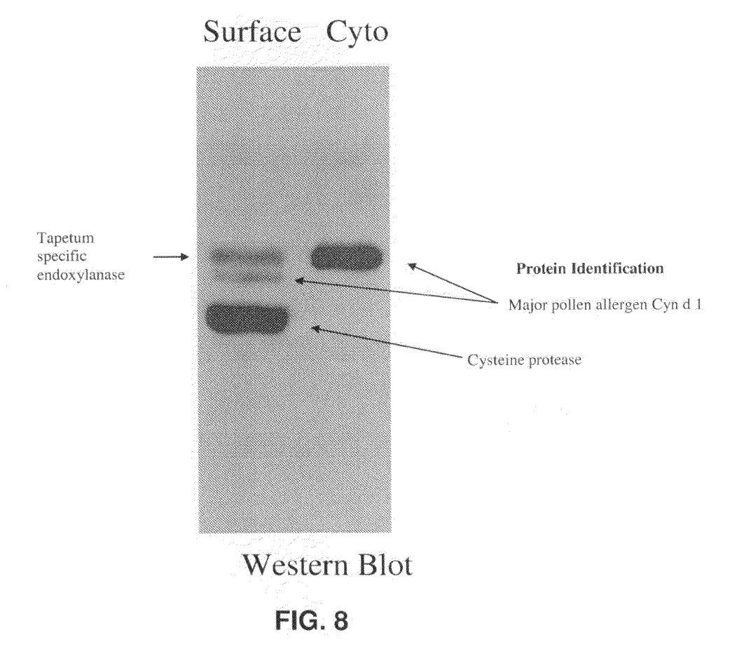

This example demonstrates that cytoplasmic and surface fractions of pollen extracts obtained using methods disclosed herein were used to identify specific pollen allergens that cross-react with antibody sera from individuals. Protein fractions were isolated from the cytoplasmic portions and the surface portion of non-defatted Bermuda grass pollen. Sufficient proteinaceous material from the pollen fractions were analyzed by polyacrylamide gel electrophoresis followed by western blot with pooled sera or stained with Coomassie or other suitable reagents (FIG. 7). Excision of the band, followed by digestion with specific proteases yielded peptides that were sequenced using techniques known to those of skill in the art, e.g., peptide fingerprinting on a MALDI-TOF analyzer. After obtaining peptide sequences, GenBank and other relevant databases were searched to identify candidate genes or ESTs. The candidate genes include major pollen allergen Cyn d 1, tapetum specific endoxylanase, and cysteine protease from Bermuda grass pollen (FIG. 8). Some of the peptide sequences analyzed are shown in TABLE 1.

TABLE 1

Peptide sequences of pollen allergens.

| Pollen | Short Peptide | GenBank | Primer sequences to |
|---|---|---|---|
| Allergen | Sequences | Ac. No. | amplify DNA sequence |
| Cysteine | Best Match | AY112580 | Forward: |
| protease | YWLVK | (Zea mays) | 1EF0 - 5′ CGACTGCGACCCCTACGAC 3′ |
| (~20 kDa) | SKGAVTPIK | | 1EF1 - 5′ CGACTGCGACCCCTACGACG 3′ |
| | PTTVMAWFILVPHCPEK | | 1EF2 - 5′ CGACTGCGACCCCTACGACGG 3′ |
| | | | 1EF3 - 5′ CGACTGCGACCCCTACGACGGC 3′ |
| | Other Matches | AK068469 | |
| | KGSTSVK | (Oryza | |
| | KQIMWSELS | sativa) | Reverse: |
| | AVWSALSTGEKQQR | | 1ER0 - 5′ TCTGCCCCCACGAGTTCTT 3′ |
| | | | 1ER1 - 5′ TCTGCCCCCACGAGTTCTTG 3′ |
| | | | 1ER2 - 5′ TCTGCCCCCACGAGTTCTTGA 3′ |
| | VVGGGGAVRGR | CK162 | 1ER3 - 5′ TCTGCCCCCACGAGTTCTTGAC 3′ |
| | | (Triticum | |
| | | aestivum) | |
| Xylanase | WFTSR | AY110911 | Forward: |
| (~30 kDa) | YLEQVLR | (Zea mays) | 1XF0 - 5′ CGGCATACGAGAAGTGGTTC 3′ |
| | NLPVGDLVDK | | 1XF1 - 5′ CGGCATACGAGAAGTGGTTCA 3′ |
| | | | 1XF2 - 5′ CGGCATACGAGAAGTGGTTCAC 3′ |
| | | | 1XF3 - 5′ CGGCATACGAGAAGTGGTTCACG 3′ |
| | | | 1XG0 - 5′ AGTGGACCCAGAACCATGAG 3′ |
| | | | 1XG1 - 5′ AGTGGACCCAGAACCATGAGG 3′ |
| | | | 1XG2 - 5′ AGTGGACCCAGAACCATGAGGA 3′ |
| | | | 1XG3 - 5′ AGTGGACCCAGAACCATGAGGAC 3′ |
| | | | Reverse: |
| | | | 1XS0 - 5′ CTGTTGTTCGTGAGGCACAT 3′ |
| | | | 1XS1 - 5′ CTGTTGTTCGTGAGGCACATG 3′ |
| | | | 1XS2 - 5′ CTGTTGTTCGTGAGGCACATGA 3′ |
| | | | 1XS3 - 5′ CTGTTGTTCGTGAGGCACATGAC 3′ |
| | | | 1XR0 - 5′ TGAAGCTGTTGTTCGTGAGG 3′ |
| | | | 1XR1 - 5′ TGAAGCTGTTGTTCGTGAGGC 3′ |
| | | | 1XR2 - 5′ TGAAGCTGTTGTTCGTGAGGCA 3′ |
| | | | 1XR3 - 5′ TGAAGCTGTTGTTCGTGAGGCAC 3′ |

Full length cDNAs or genomic fragments or complete peptide sequences corresponding to the peptide sequences identified herein for the candidate genes shown in FIG. 8 are obtained using techniques known to one of ordinary skill in the art. Degenerate PCR primers that correspond to the sequenced peptides are used to amplify the corresponding genes from a cDNA library or a genomic library. Subsequently, 5′ and 3′ RACE experiments are used to characterize the full-length message. The peptide sequence and the allergenic epitope sequence are identified through any suitable technique known to those of ordinary skill in the art.

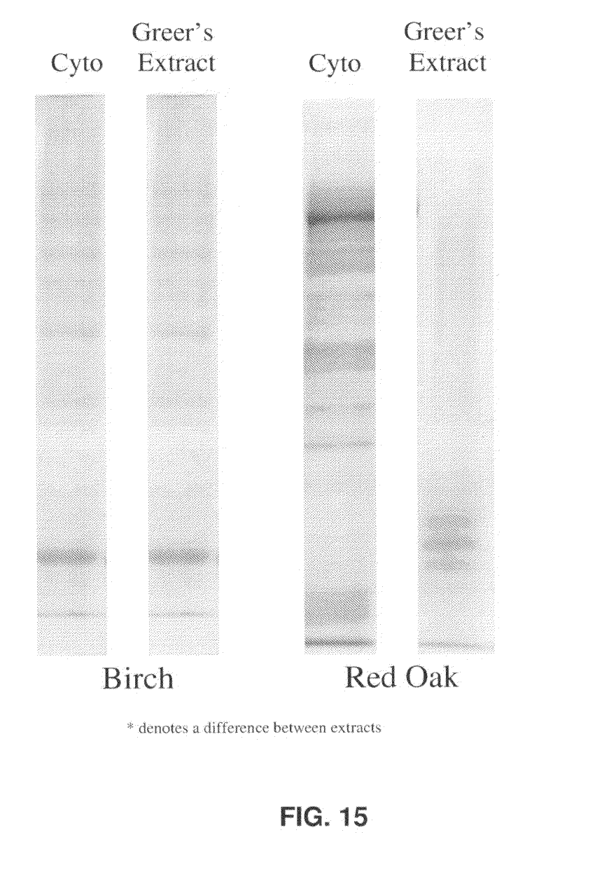

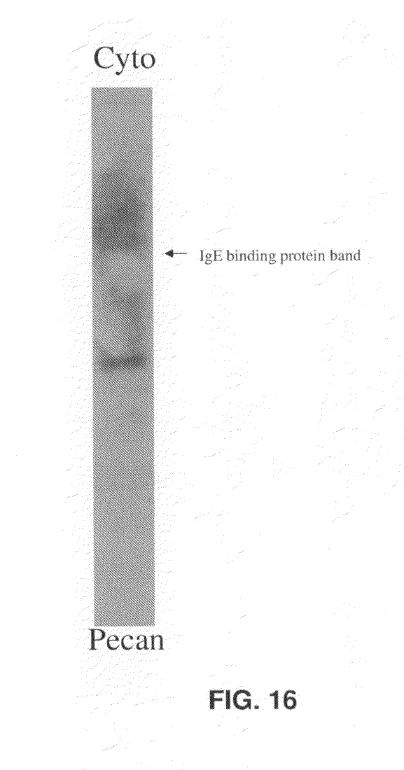

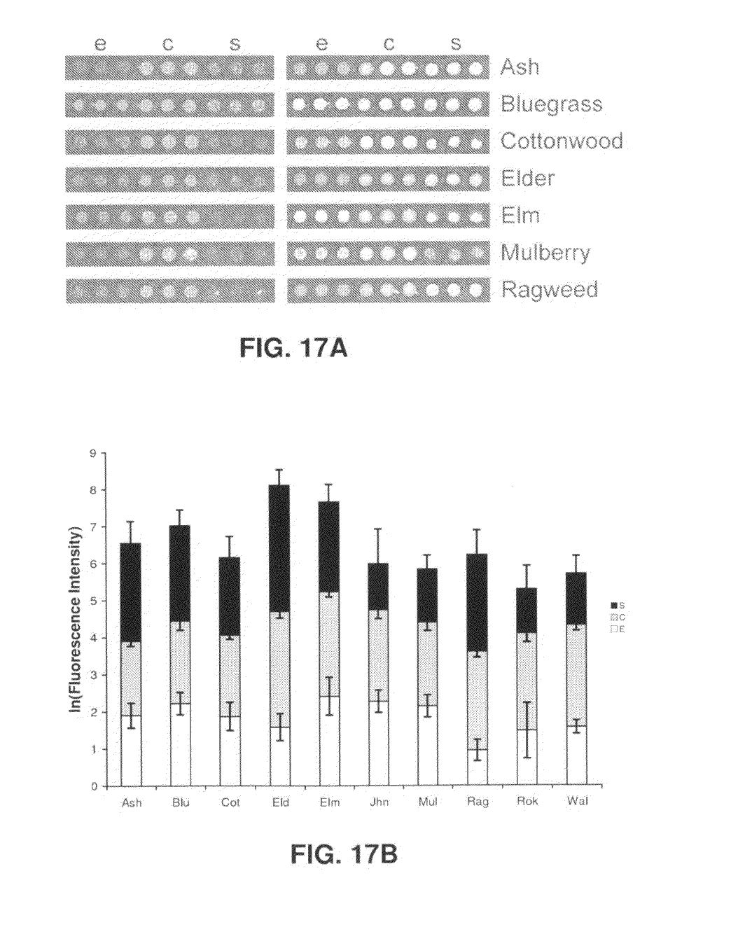

Similarly, allergens can be identified from extracts obtained from other plant species and also from other commercially available extracts. For example, FIG. 15 shows comparison of pollen cytoplasm extracts isolated by the methods disclosed herein with a commercially available pollen extract (Greer Laboratories, Lenoir N.C.) using polyacrylamide gel electrophoresis for birch and red oak species and FIG. 16 shows IgE binding protein band from a pollen extract of Pecan. Polyacrylamide gel electrophoresis (PAGE) of pollen extracts isolated using protocols disclosed herein revealed specific differences with the commercially available extracts, demonstrating the need for improved isolation procedures for pollen components that are disclosed herein, e.g., isolation of pollen components from cytoplasmic and surface fractions of non-defatted pollen.

### Example 10

### Hyposensitization or Immunotherapy with Allergenic Pollen

### Components

Hyposensitization or allergy desensitization is an immunotherapy where the patient is desensitized to a particular allergen or a group of allergen by administering progressively higher doses of the allergen of interest. This procedure can either reduce the severity of the allergy response or eliminate hypersensitivity and relies on the progressive skewing of IgG (“the blocking antibody”) production, as opposed to the excessive IgE production seen in hypersensitivity type I cases. It is believed that in allergic reaction the body responds to harmless substances from the environment as if they were invading parasites. The body begins to produce specific immunoglobulin of the E class, IgE. It appears that allergy shots increases the amount of a different class of immunoglobulins, called IgG. It is believed that when IgG molecules circulate in the blood plasma and tissue fluids in large amounts, IgGs bind to allergens and reduce the ability of IgE to detect the presence of the allergens. Thus, the inflammation, secretions, and tissue alterations that take place in untreated allergic disease decrease with immunotherapy. The relative increase of the IgG to IgE ratio results in better tolerance towards the allergen. By giving small but increasing amounts of allergen at regular intervals, tolerance increases and the individual becomes “immune” to the allergens and can tolerate them with reduced symptoms.

Sera from an allergy sufferer or a patient is screened with the diagnostic pollen microarray disclosed herein. An allergy profile of the patient is obtained that quantitatively shows specific allergenic response. Depending on the allergy profile, specific pollen components are identified for hyposensitization or allergy therapy. Small hypodermic syringes are used to inject allergen extracts. Injections are usually given into the loose tissue over the back of the upper arm, half way between the shoulder and elbow. Injections are given under the skin (“subcutaneous”). A suitable range of dosage for hyposensitization experiments or treatments is in the range of about 0.001 microgram to about 1 milligram or 0.01 microgram to 100 microgram. Allergen concentration or dosage depends on the nature of the allergenic response of the patient, which can be evaluated from the allergy profile and also the tolerance exhibited by the individual. Allergy injections are started at very low doses. The dose is gradually increased on a regular (and usually weekly) basis, until a “maintenance” dose is reached. This generally translates to four to six months of weekly injections to reach the maintenance dose. After the maintenance dose is reached, the injections are administered less often (every two to four weeks) on a regular basis. Maintenance injections are normally given once per month for a few years.

### MATERIALS AND METHODS

A. Extraction of components from unwashed pollen-pollen coat purification protocol. 5.0 g of dry pollen (stored at −20° C.) was measured into a 50 mL conical tube. The pollen material was covered with 6 mL cyclohexane and vortexed for 5 minutes. The sample was spun in a low speed centrifuge at 3000 rpm/4° C. for 5 minutes or filtered. The supernatant was removed and transferred to new tube kept on ice. The supernatant was washed with 4 mL cyclohexane and vortexed for 5 minutes and was spun for 5 minutes. Both the supernatants were combined for further analysis. The cyclohexane steps were repeated 6 times until the supernatant is clear. The remaining pollen material was preserved for extracting the cytoplasmic fraction. The combined supernatant was spun to remove any remaining pollen and the supernatant was transferred to a fresh tube.

The cyclohexane was evaporated by passing air over it, in the fume hood until about 1 ml of cycleohexane is left. About 1 ml of the remaining cyclohexane was transferred into a 1.5 ml Eppendorf™ tubes and was evaporated until about 0.5 ml cyclohexane remains. An equal volume of 20% TCA (trichloroacetic acid) or 80% acetone was added to the protein sample. The sample was vortexed for 30 minutes at 4° C. The Eppendorf™ tubes were spun in a microfuge at 14,000 rpm at 4° C. for 15 minutes. All the samples were maintained on ice. The supernatant was carefully removed and the pellet was air dried. The pellet was resuspended in a suitable buffer and the pH of the final sample was adjusted to about pH 7.0.

B. Extraction of components from washed pollen. Cyclohexane-washed pollen pellet was suspended in TBS-T (20 mM Tris, 136 mM NaCl, 0.1% Tween 20, pH 7.5). The suspended pellets were frozen in liquid nitrogen and were thawed immediately. This step was repeated twice. The sample was then transferred to a mortar and was ground with a pestle consistently for 7 minutes. The residue was transferred to centrifuge tubes and spun at 14,000 rpm for 5 minutes in a centrifuge or filtered. The supernatant was transferred to another tube and 1:100 protease inhibitors were added and the samples were stored at 4° C. Equal volumes of 20% TCA or 80% acetone were added to the samples. The samples were incubated for about 30 minutes on ice and were spun for 15 minutes at 4° C. The supernatant was removed and the pellet was washed with 100% acetone. The washed pellet was incubated on ice for 10 minutes was then spun for 5 minutes at 4° C. The supernatant was removed and the pellet was stored at 4° C. until the next step. The pellet was resuspended in a suitable buffer and the pH of the final sample was adjusted to about pH 7.0.

C. Preparation of low-density arrays. Proteins were quantified using a suitable assay kit (such as Coomassie Plus™-The Better Bradford Assay Kit, catalog #23236, Pierce, Rockford, Ill.). About 2 μg of pollen components were spotted in duplicates onto nitrocellulose membrane. Also about spot 2 μl of undiluted serum and 1 μl of undiluted 2° Ab were spotted in duplicates as positive controls. The filter was washed with TBS-T(20 mM Tris, 136 mM NaCl, 0.1% Tween 20, pH 7.5) for 15 minutes. The filter was incubated at 20° C. for 1 hour in blocking buffer (5% nonfat dried milk or 1% bovine serum albumin in TBS-T). The filter was incubated overnight at 4° C. in the same buffer along with the addition of antisera (diluted 1:2 to 1:100, as necessary). After incubation, the filter was washed with TBS-T, 3 times for 5 minutes each. A secondary antibody, such as goat anti-human IgE conjugated to HRP (Catalog #48-139-H, Antibodies Incorporated, Davis, Calif.) was added at a suitable dilution (1:100 to 1:1000) and was incubated for about 1 hour at room temperature in TBS-T or blocking buffer. The membrane was washed with TBS-T, 3 times for 15 minutes each. Bound antibodies were detected using an ECL kit (Amersham Life Science, catalog #1059243 and #1059250) according to the manufacturer's instructions.

D. High-density array. Proteins were quantified using a suitable assay kit (such as Coomassie Plus™-The Better Bradford Assay Kit, catalog #23236, Pierce, Rockford, Ill.). About 1.5 nl of pollen components dissolved at 0.3-1 μg protein/μl into Protein Printing Buffer (#PPB, ArrayIt, Sunnydale, Calif.) were spotted in triplicates using NanoPrint™ Microarray System onto SuperEpoxy™ slides with about 1.5 nL drop size. Serial dilutions (1 pg-800 pg) of purified IgE (IgE Myeloma Serum, catalog #30-AI05, Fitzgerald Industries International, Concord, Mass.) IgG, (Intact Human IgG, catalog #P80-105, Bethyl Laboratories Inc., Montgomery, Tex.), and IgA (Human IgA, catalog #P80-102, Bethyl Laboratories Inc., Montgomery, Tex.) as well as 1.5 nL of Human Serum Albumin (catalog# 05420-500 MG, Sigma, St. Louis, Mo.), and Bet v 1, Phl p 2, Amb a 1, Alt a 1, and Der p 1 (Biomay, Vienna, Austria) dissolved at 0.3-1.0 μg/μL were spotted in triplicates. Slides were washed with 1×PBS-T on SpeciMix™ for 10 minutes 3 times (reagent: 3×5 mL PBS-T). Non-specific binding was limited by treating slides with BlockIt™ buffer on SpeciMix™ for 120 minutes (reagent: 3 mL BlockIt). Slides were rinsed with 1×PBS-T on SpeciMix™ for 5 minutes 5 times (reagent: 5×5 mL PBS-T). Slides were incubated with human sera in ProPlate™ using 1:1 dilutions for 60 minutes @ room temperature, overnight @ 4° C. Sera should be diluted in PBS-T containing 1% HSA (reagent: 48 μL PBS-T with 1% HSA+sera/well). Be sure not to cross-contaminate from well to well. Sera was aspirated and PBS-T was added to each well 3× as an initial wash (reagent: 3×100 μL PBS-T per well). Slides were removed from ProPlate™ and washed with 1×PBS-T SpeciMix™ for 10 minutes 3 times (reagent: 3×5 mL PBS-T). Slides were incubated with 2° Ab on SpeciMix™ using 1:50 (IgE) and 1:100 (IgG) dilutions. Antibody should be diluted in PBS-T containing 1% HSA for 120 minutes (reagent: 2 mL PBS-T with 1% HSA+2° Ab). Slides were washed with PBS-T on SpeciMix™ for 10 minutes two times (reagent: 2×5 mL PBS-T) and with PBS on SpeciMix™ for 10 minutes twice (reagent: 2×5 mL PBS). Slides were rinsed with ddH₂O for a few seconds and dried by spinning in low speed centrifuge and stored in the dark until scanning. Slides are scanned with GenePix™ 4000B and images are converted into data using GenePix™ Pro 6.0.1 software.

E. Mapping pollen allergy genes. Sera from individual patients are hybridized to pollen arrays, and bound antibodies are detected with anti-IgE, anti-IgG, or anti-IgA, as described herein. Using a microarray scanner, fluorescent signals corresponding to each spot on the hybridized array are calculated. For each signal above a background threshold, a quantitative score to indicate patient sensitivity is assigned. Each score, or groups of scores, constitutes a sensitivity phenotype.

For individuals surveyed, genotype data (SNPs, microsatellies, indels) are collected corresponding to informative markers distributed across the genome. The LOD-based linkage analysis of families is used to identify regions in the genome that correlate with sensitivity phenotypes. In some cases, more specific mapping methods, including homozygosity by descent mapping, can be used. When useful, association mapping methods are employed to narrow regions of interest and identify alleles that correlate with the phenotype of interest.

F. Protocol for Method of Identifying Novel (Pollen) Allergens: One of the first steps is to isolate proteins from desired tissues, e.g., from pollen cytoplasm or pollen surface. Isolated and purified protein fractions are electrophoresed on a separating gel (Poly Acrylamide Gel Electrophoresis), preferably in duplicates. Then, one of the gels is stained with a visualizing agent, for example, Coomassie, Amido Black, Sypro Red, or any suitable visualizing or labeling agent or dye. The electrophoresed fractions in the second gel are transferred on to a blotting membrane as in a standard western blotting procedure disclosed herein. The membrane is then probed with pooled human sera as the primary antibody in 1:1 antibody:blocking agent mixture. The primary antibody-bound membrane is then hybridized with anti-human IgE conjugated to a visualizing agent (colorimetric, chemiflourescent, or chemiluminescent), in a ratio appropriate to the blot size (e.g., 1:20 antibody:blocking agent is suitable). The hybridization reaction is visualized after developing an exposed film to detect the fluorescent or radioactive or chemiluminescent reactions. The reactive bands on the blot are matched to the corresponding ones on the gel. The bands from the gel are cut out and sequences are analyzed from the purified peptide product. The resulting sequences are compared against other proteins in the database to obtain a preliminary understanding as to their structure and function.

G. Western Blot Protocol: Gel to Membrane Blotting: 1. Whatman™ filter papers and one Immobilon-P (PVDF) membrane were cut according to the size of the gel. Immobilon-P membrane was soaked in 100% methanol for 15s and then was transferred to ddH₂O for 2 min followed by equilibration in semi-dry transfer buffer (sdTB) for 5 min. The gel was removed from plate sandwich noting the appropriate left to right orientation. The gel was rinsed briefly in a semi-dry transfer buffer (sdTB). sdTB-wetted filter papers were layered as a platform of blotter. The immobilon-P membrane was positioned on top noting the orientation to match the gel orientation. The gel was placed in known orientation onto Immobilon-P membrane. More wetted filters papers were placed on top and the air bubbles were removed by rolling a smooth surfaced object. The top of the electro-blotter was installed and the system was run at 150 mA for 45 min. The filter papers were removed and the sizes of the standards were checked to confirm blotting. The gel was removed and slits in the membrane were cut at 75, 50, 25, and 10 kDa.

(i) Probing the Membrane: The transferred membrane was rinsed in TBSt (0.05% Tween 20 in TBS) for about 5 min. The membrane was blocked 10-20 ml of blocking solution for at least one hour at room temp with gentle agitation. Blocking Solution: 3% BSA in TBSt buffer. The blocking solution was decanted and the membrane was washed with TBSt for 5 min with gentle agitation. The wash solution was decanted and the tubes were incubated with primary antibody (diluted in blocking Solution 1:1) overnight at 4° C. with gentle agitation. Primary antibody solution was decanted and the membrane was washed in TBSt for 5 min. The washes were repeated twice for a total wash time of 15 min. The wash solution was decanted and conjugated secondary antibody was added that was, diluted in blocking solution 1:20. The system was incubated for 1 hr with gentle agitation. The conjugate solution was decanted and the system was washed in TBSt for 5 min. This step was repeated and a final 5 min-wash was performed in TBS to remove residual detergent.

(ii). Developing the Membrane: Equal parts of ECL (chemiluminescent) solutions A and B (about 1 ml each per membrane) were added. ECL solution mixture was spread onto protein side of membrane and was incubated for 5 min The filter was drained and wrapped in a plastic wrap and was exposed to X-ray film. Exposure times ranged from 30 sec to 10 min. The exposed films were developed in a developer.

TABLE 2
Reproducibility of microarray testing of allergen-specific IgE
Semi-dry Transfer Buffer (Bjerrum and Schafer-Nielsen)

| Concentration | For 1 liter add |
|---|---|
| 48 mM Tris | 5.82 g |
| 39 mM glycine | 2.93 g |
| 0.0375% SDS or | 0.375 g or 3.75 ml of 10% SDS |
| 20% MeOH | 200 ml |
| ddH₂O | Add to 1 liter final volume |

Commercial Extracts

| Reproducibility | Grasses | Weeds | Trees | Other |
|---|---|---|---|---|
| Intra-slide | 0.1 | 0.12 | 0.1 | 0.13 |
| Inter-slide | 0.15 | 0.12 | 0.14 | 0.17 |
| Inter-assay | 0.14 | 0.11 | 0.34 | 0.14 |

| | | | |
|---|---|---|---|
| Reproducibility | Grasses | Weeds | Trees |

In-Lab Cytoplasm

| | | | |
|---|---|---|---|
| Intra-slide | 0.26 | 0.19 | 0.21 |
| Inter-slide | 0.22 | 0.17 | 0.19 |
| Inter-assay | 0.38 | 0.12 | 0.14 |

In-Lab Surface

| | | | |
|---|---|---|---|
| Intra-slide | 0.15 | 0.13 | 0.28 |
| Inter-slide | 0.35 | 0.22 | 0.31 |
| Inter-assay | 0.36 | 0.19 | 0.21 |

Median coefficient of variation (CV: % standard deviation of mean) for 6 high-IgE individuals tested at serum concentration of 25% broken down by allergen family. Grasses are bermuda grass, bluegrass, johnson grass, orchard grass, ryegrass, and timothy grass. Weeds are mugwort and ragweed. Trees are alder, ash, birch, elder, cottonwood, elm, mulberry, red oak, white oak, olive, pecan, sycamore, walnut, and cedar. Other are Alternaria alternana, Aspergillus niger, German cockroach, American cockroach, Dust, Cat, Dog, Der p, and Der f.

### DOCUMENTS

Abney, M., C. Ober, and M. S. McPeek, Quantitative-trait homozygosity and association mapping and empirical genomewide significance in large, complex pedigrees: fasting serum-insulin level in the Hutterites. Am J Hum Genet, 2002. 70(4): p. 920-34.
Bollag, D., M. Rozycki, and S. Edelstein, Protein Methods. 1996, New York: Wiley-Liss. 415.
Christie, W. W. (1992). Detectors for high-performance liquid chromatography of lipids with special reference to evaporative light-scattering detection. In: ‘Advances in Lipid Methodology—One’ (edited by W. W. Christie, Oily Press, Ayr), pp. 239-271.
Dennison, C., A guide to protein isolation. Focus on Structural Biology, 2003, Boston: Kluwer Academic Publishers. 248.
Mattner et. al., (2005). Exogenous and endogenous glycolipid antigens activate NKT cells during microbial infections., Nature, 2005; 434 (7032): 525-9.
Mayfield, J. A., et al., Gene families from the Arabidopsis thaliana pollen coat proteome. Science, 2001. 292(5526): p. 2482-5.
Moreau, R. A. (1993). Quantitative analysis of lipids by HPLC with a flame-ionization detector or an evaporative light-scattering detector. In: ‘Lipid Chromatographic Analysis’, pp. 251-272 (edited by T. Shibamoto, Dekker, N.Y.).
Ogren, T. L., The Revolutionary Guide to Healthy Landscaping, Ten Speed Press, (Jun. 1, 2000).
Strachan, T. and A. Read, Human Molecular Genetics 2. 2nd ed. 1999, New York: Wiley-Liss. 576.

1. An IgE-reactive cysteine protease from pollen.

2. The IgE-reactive cysteine protease of claim 1 wherein the pollen is from Bermuda grass with an amino acid sequence having 80%, preferably 90-95% homology to the amino acid sequences as in FIG. 10 and/or FIG. 14 encoded by an DNA sequence having 80%, preferably 90-95% homology to the mRNA sequences as in FIG. 10.

3. The IgE-reactive cysteine protease of claim 1 wherein the pollen is from Johnson grass with an amino acid sequence having 80%, preferably 90-95% homology to the amino acid sequences as in FIG. 13 and/or FIG. 14 and/or encoded by an DNA sequence having 80%, preferably 90-95% homology to the mRNA sequences as in FIG. 13 and/or FIG. 14.

4. Use of a cysteine protease of claim 1 to treat allergy by desensitizing allergic subjects to purified, recombinant cysteine protease.

5. Use of an inhibitor of a cysteine protease of claim 1 to prevent the symptoms of allergy or asthma in a subject.

6. A method of constructing a pollen allergen array from specific species, the method comprising the steps of
(a) isolating pollen from the specific species;
(b) extracting pollen components using organic solvents; and
(c) affixing pollen components to a solid support.

7. The method of claim 6 wherein the specific species are selected from the group consisting of Wal (Walnut), Ber (Bermuda grass), Syc (Sycamore), Orc (Orchard grass), Pec (Pecan), Tim (Timothy grass), Olv (Olive), Mug (Mugwort), WOk (White Oak), Rag (Ragweed), Ald (Alder), Eld (Box Elder), Cot (Cottonwood), Mul (Mulberry), Jhn (Johnson grass), Elm, Ash, Ced (Cedar), Blu (Bluegrass), Bir (Birch), Rye, and ROk (Red Oak).

8. The method of claim 6 wherein the organic solvents are selected from a group consisting of polar, non-polar, protic and aprotic solvents.

9. A method of measuring sensitivity to pollen allergens in an individual, the method comprising:
(a) providing the pollen array of claim 10 comprising at least one pollen surface allergen;
(b) applying a serum sample from the individual to the pollen array; and
(c) analyzing the sensitivity to pollen allergen by measuring antibody levels to the allergen.

10. A diagnostic pollen array comprising at least one pollen allergen on a solid support.

11. The array of claim 10 comprising a plurality of pollen allergens.

12. Use of an allergen from plant pollen for the preparation of a medicament for the treatment of allergies.

13. A composition comprising pollen allergens obtained by organic solvents from pollen.

14. The composition of claim 13, wherein the pollen is unwashed.

15. Pharmaceutical composition comprising the composition of claim 13 and a pharmaceutically acceptable carrier.

16. A method to identify new pollen allergens, the method comprising:
(a) providing a diagnostic pollen array;
(b) identifying a pollen allergen that cross-reacts with a serum sample obtained from at least one allergic individual; and
(c) obtaining sequence information of the pollen allergens.

17. The method of claim 16 wherein the sequence information is obtained through a mass-spectroscopic analysis.

18. A method to measure antibody levels to pollen allergens in an individual, the method comprising:
(a) providing a diagnostic pollen array comprising a pollen allergen;
(b) applying a serum sample from an individual to the diagnostic array; and
(c) measuring antibody levels to the pollen allergen by measuring antibody-allergen reactions on the array.

19. A method to develop a pollen-specific allergy treatment in an individual, the method comprising:
(a) providing a diagnostic pollen array comprising a pollen allergen;
(b) identifying a pollen allergen that contributes to allergy in the individual by the method of claim 16; and
(c) developing a therapy to treat allergy to the pollen allergen in the individual.

20. A method to hyposensitize an individual to one or more specific pollen allergens, the method comprising:
(a) providing a diagnostic pollen array of claim 10 comprising at least one pollen allergen to develop an allergy profile of the individual;
(b) administering one or more pollen allergens identified in (a) at a dosage that is sufficient to elicit a desensitization response in the individual; and
(c) hyposensitizing the individual by administering progressively higher doses of allergen and monitoring the hyposensitization response in the individual to the pollen allergen using the diagnostic pollen array.

21. A method of identifying a genetic locus that contributes to allergy, the method comprising:
(a) diagnosing allergen sensitization in a group of individuals using the diagnostic pollen array of claim 10;
(b) correlating results of the diagnosing to at least one genetic marker at a locus; and identifying the genetic locus that contributes to allergy.

22. Pharmaceutical composition comprising the composition of claim 14 and a pharmaceutically acceptable carrier.
